SEQUENCE LISTING (40 sequences)

SEQ ID NO: 1 - DNA, 20 bp, Homo sapiens
accgaattcgattcgatccg

SEQ ID NO: 2 - DNA, 20 bp, Homo sapiens
gaccgaattcgattcgatcc

SEQ ID NO: 3 - DNA, 20 bp, Homo sapiens
cgaccgaattcgattcgatc

SEQ ID NO: 4 - DNA, 20 bp, Homo sapiens
ccgaccgaattcgattcgat

SEQ ID NO: 5 - DNA, 20 bp, Homo sapiens
tccgaccgaattcgattcga

SEQ ID NO: 6 - DNA, 20 bp, Homo sapiens
atccgaccgaattcgattcg

SEQ ID NO: 7 - DNA, 20 bp, Homo sapiens
gatccgaccgaattcgattc

SEQ ID NO: 8 - DNA, 20 bp, Homo sapiens
cgatccgaccgaattcgatt

SEQ ID NO: 9 - DNA, 20 bp, Homo sapiens
tcgatccgaccgaattcgat

SEQ ID NO: 10 - DNA, 20 bp, Homo sapiens
ttcgatccgaccgaattcga

SEQ ID NO: 11 - DNA, 20 bp, Homo sapiens
attcgatccgaccgaattcg

SEQ ID NO: 12 - DNA, 20 bp, Homo sapiens
gattcgatccgaccgaattc

SEQ ID NO: 13 - DNA, 20 bp, Homo sapiens
cgattcgatccgaccgaatt

SEQ ID NO: 14 - DNA, 20 bp, Homo sapiens
tcgattcgatccgaccgaat

SEQ ID NO: 15 - DNA, 20 bp, Homo sapiens
ttcgattcgatccgaccgaa

SEQ ID NO: 16 - DNA, 20 bp, Homo sapiens
attcgattcgatccgaccga

SEQ ID NO: 17 - DNA, 20 bp, Homo sapiens
aattcgattcgatccgaccg

SEQ ID NO: 18 - DNA, 20 bp, Homo sapiens
gaattcgattcgatccgacc

SEQ ID NO: 19 - DNA, 20 bp, Homo sapiens
cgaattcgattcgatccgac

SEQ ID NO: 20 - DNA, 20 bp, Homo sapiens
ccgaattcgattcgatccga

SEQ ID NO: 21 - DNA, 20 bp, Homo sapiens
gtgatagtgatacggatccg

SEQ ID NO: 22 - DNA, 20 bp, Homo sapiens
agtgatagtgataccgatcc

SEQ ID NO: 23 - DNA, 20 bp, Homo sapiens
aagtgatagtgatatcgatc

SEQ ID NO: 24 - DNA, 20 bp, Homo sapiens
gaagtgatagtgatttcgat

SEQ ID NO: 25 - DNA, 20 bp, Homo sapiens
cgaagtgatagtgaattcga

SEQ ID NO: 26 - DNA, 20 bp, Homo sapiens
acgaagtgatagtggattcg

SEQ ID NO: 27 - DNA, 20 bp, Homo sapiens
tacgaagtgatagtcgattc

SEQ ID NO: 28 - DNA, 20 bp, Homo sapiens
atacgaagtgatagtcgatt

SEQ ID NO: 29 - DNA, 20 bp, Homo sapiens
gatacgaagtgatattcgat

SEQ ID NO: 30 - DNA, 20 bp, Homo sapiens
tgatacgaagtgatattcga

SEQ ID NO: 31 - DNA, 20 bp, Homo sapiens
gtgatacgaagtgaaattcg

SEQ ID NO: 32 - DNA, 20 bp, Homo sapiens
agtgatacgaagtggaattc

SEQ ID NO: 33 - DNA, 20 bp, Homo sapiens
tagtgatacgaagtcgaatt

SEQ ID NO: 34 - DNA, 20 bp, Homo sapiens
atagtgatacgaagccgaat

SEQ ID NO: 35 - DNA, 20 bp, Homo sapiens
gatagtgatacgaagccgaa

SEQ ID NO: 36 - DNA, 20 bp, Homo sapiens
tgatagtgatacgaagccga

SEQ ID NO: 37 - DNA, 20 bp, Homo sapiens
gtgatagtgatacgaagccg

SEQ ID NO: 38 - DNA, 20 bp, Homo sapiens
agtgatagtgatacgaagcc

SEQ ID NO: 39 - DNA, 20 bp, Homo sapiens
aagtgatagtgatacgaagc

SEQ ID NO: 40 - DNA, 20 bp, Homo sapiens
taagtgatagtgatacgaag

(19) United States
(12) Patent Application Publication
(10) Pub. No.: US 2007/0031856 A1
(43) Pub. Date: Feb. 8, 2007

(54) Biodisc microarray and its fabrication, use, and scanning

(76) Inventor: Gilbert Hong, San Jose, CA (US)

Correspondence Address: VIERRA MAGEN MARCUS & DENIRO LLP, 575 MARKET STREET SUITE 2500, SAN FRANCISCO, CA, 94105, US

(21) Appl. No.: 11/339,875

(22) Filed: Jan. 26, 2006

Related U.S. Application Data
(63) Continuation-in-part of application No. 10/412,973, filed on Apr. 14, 2003.
(60) Provisional application No. 60/434,505, filed on Dec. 20, 2002.

(51) Int. Cl. C12Q 1/68; C12M 1/34

(52) U.S. Cl. 435/6; 435/287.2

(57) ABSTRACT

A biodisc comprises a CD-type optical disc with small feature oligonucleotide probes disposed on its surfaces. The biodisc's probes are either custom-fabricated in-situ with a master-duplicate tandem arrangement of discs that allows one disc and a reading/tracking head to control the probe locations being synthesized pass-by-pass on the duplicate. Or the biodisc is mass-produced in a manufacturing process that includes a spin-on-and-peel (SOAP) method with nickel master for standardized biodisc oligonucleotide probes. In one embodiment of the invention, such biodisc is fabricated with four masks only, and these are shifted between depositions to synthesize particular individual 4-mer+ nucleotide probes.

This application is a continuation-in-part of application Ser. No. 10/412,973 filed on Apr. 14, 2003, entitled “Biodisc Microarray And Its Fabrication, Use, And Scanning”, which claims benefit of Provisional Application Ser. No. 60/434,505 filed on Dec. 20, 2002 entitled “Optical Disk For Nucleic Acid Target Sequencing”.

### BACKGROUND OF THE INVENTION

1. Field of the Invention

The present invention relates to microarrays of oligonucleotide probes, and more particularly to the fabrication, use, and reading of microarrays disposed on compact disc type substrates that are rotationally scannable in optical media computer disc drives.

2. Description of the Related Art

DNA microarrays are glass micro slides or plastic plates with genomic DNA, cDNA, oligonucleotides, or other DNA samples in ordered two-dimensional matrices. Biochip DNA microarrays use oligonucleotides and measure the length of each probe's sequences in “mers”. Typical biochip oligonucleotides probes are 20 mers in length. Probes or various lengths are designed for various applications to optimize cost and/or sensitivity and specificity.

DNA microarrays are used to analyze gene expression and genomic clones or to detect mutations, single nucleotide polymorphisms (SNPs). The microarray DNA selected is often from a group of related genes such as those expressed in a particular tissue, during a certain developmental stage, in certain pathways, or after treatment with drugs or other agents. Expression of that group of genes is quantified by measuring the hybridization of fluorescent-labeled RNA or DNA to the known DNA sequences on the chip. Transcriptional changes can be monitored by gene expression profiling through organ and tissue development, microbiological infection, and tumor formation.

DNA samples are now being automatically analyzed with so-called DNA microarray chips. Such semiconductor chips are able to sense where fluorescent-dye marked DNA fragments have attached themselves to cells arranged on the chip. Each cell has an address and a fragment of a DNA, also known as oligomers. Samples with unknown fragments of DNA material are floated by the chip surface. These will attach themselves, in a natural DNA-DNA-pairing process called hybridization, at various cell addresses of the DNA microarray chip. The DNA sequences of the sample material fragments will be revealed by chip addresses where each fragment attaches itself. The sample material fragments are therefore prepared with fluorescent dye to later make their address-of-attachment optically visible and readable by the pickup head.

There are a very large number of genetic sequences possible. Far too many to fit on a single microarray. So conventional practice is to engineer microarrays that have those genetic sequences that are likely to be useful in a particular line of research. Such microarrays are either purchased as standard models from a catalog, or custom-made as the need arises.

Agilent Technologies, Inc. uses its inkjet technology to print oligos and whole cDNAs onto glass slides. The non-contact inkjet technology produces microarrays with more uniform and consistent features. The number of features depends on the type of microarray. Up to 16,200 features can be microarrayed on Agilent's cDNA catalog microarrays. Of these, there are a number of control features, and orientation markers. The Human-1 cDNA Microarray includes 13,675 individual, microarrayed clones in addition to a series control genes and orientation markers. The inkjet technology is low cost and yet low resolution. The current application attempt to address the need for higher capacity and higher density than achievable with inkjet method.

Biologically relevant Deoxyribose nucleic acids (DNA) basically consists of four bases, adenine (A), guanine (G), cytosine (C) and thymine (T). A given sequence of bases such as ATTGCATGA will bind its complementary strand TAACGTACT to form a stable duplex. Thus biological information is stored in a one dimensional sequence of bases.

Sequencing by hybridization (SbH) is a sequence analysis technology that exploits the natural base pairing to decode the nitrogenous bases of assays of interest. Prior art devices attach synthetic DNA base sequences to substrates to form “probes” which will hybridize to complementary base sequences in a sample “target” DNA fragment. See Mitchell D. Eggers, et al., “Genosensors, micro fabricated devices for automated DNA sequence analysis,” SPIE Vol. 1891, Advances in DNA Sequencing Technology (1993), pp. 113-126. Probes can be made in different lengths with different numbers of bases. For example, all of 65,536 different 8-base probes will be needed to detect all the possible complementary 8-base sequences that can occur anywhere in a target sample. The particular 8-base probes that hybridize the target sample will tell which particular 8-base sequences occur. Target samples longer than 8-bases can be completely read by combining 8-base probes according to their overlapping patterns. For example, a probe match of two 8-base sequences, ATTTCGGA and TTTCGGAG, can indicate a 9-base sequence in a target of ATTTCGGAG.

A particular oligonucleotide probe array system is marketed by Affymetrix, Inc. (Santa Clara, Calif.) under the trademark GENECHIP. Various oligonucleotide patterns are arranged on the probe in engineered sequences. Agilent Technologies, Inc. makes a scanner for the GENECHIP system that can read the fluorescent glows from target fragments that bind at the surface of the probes.

Special application software then interprets what nucleic acid sequences were present in the target from both the X,Y location of the light that was read from the probe and the light's relative intensity. The typical probe is organized as a flat two-dimensional array with X,Y addresses. Target nucleic acids are fluorescently labeled for hybridization to ligands with X,Y addresses in the probe arrays. See, Peter M. Goodwin, et al., “DNA sequencing by single-molecule detection of labeled nucleotides sequentially cleaved from a single strand of DNA,” SPIE Vol. 1891, Advances in DNA Sequencing Technology 5 (1993), pp. 127-131.

Prior art inkjet technology provides a flexible and yet and often expensive way to fabricate chip-type microarrays. But the feature density is not high, and the reading of such microarrays requires expensive scanners.

### SUMMARY OF THE INVENTION

An object of the present invention is to provide a system for reading nucleic acid sequences in biological assays of interest.

Another object of the present invention is to provide a microarray with a relatively large oligonucleotide probe capacity.

A further object of the present invention is to provide a microarray with a relatively dense oligonucleotide probe organization.

A still further object of the present invention is to provide a CD-format microarray that can be scanned with inexpensive CD-type optical disc drives.

Briefly, a biodisc embodiment of the present invention comprises a CD-type optical disc with small feature oligonucleotide probes disposed on its surfaces. The biodisc's probes can be custom-fabricated in-situ with a dual track arrangement. One track for global or local alignment and the other for the control of the probe locations being synthesized pass-by-pass. The biodisc can also be mass-produced in a manufacturing process that includes a spin-on-and-peel (SOAP) method with the use of a nickel or similar plastic masters for standardized biodisc oligonucleotide probes. In one embodiment of the invention, such biodisc is fabricated with four masks only, and these are shifted between depositions to synthesize particular 4-mer to 20 mer nucleotide probes.

An advantage of the present invention is that a biodisc is provided that has a dense, high population of oligonucleotide probes.

A further advantage of the present invention is that a mass production method is provided for making of an oligomer master.

Another advantage of the present invention is that a mass production method is provided for replicating of oligomer discs for DNA hybridization and assay.

Another advantage of the present invention is that a mass production method is provided for the making of biodisc hybridization cells based on similar designing features of a floppy disc or a compact disc.

A further advantage of the present invention is that a simple method and device are provided for DNA hybridization and assay.

The above and still further objects, features, and advantages of the present invention will become apparent upon consideration of the following detailed description of specific embodiments thereof, especially when taken in conjunction with the accompanying drawings.

The present invention can be accomplished using hardware, software, or a combination of both hardware and software. The software used for the present invention is stored on one or more processor readable storage media including hard disk drives, CD-ROMs, DVDs, optical disks, floppy disks, tape drives, RAM, ROM or other suitable storage devices. In alternative embodiments, some or all of the software can be replaced by dedicated hardware including custom integrated circuits, gate arrays, and special purpose computers.

These and other objects and advantages of the present invention will appear more clearly from the following description in which the preferred embodiment of the invention has been set forth in conjunction with the drawings.

### BRIEF DESCRIPTION OF THE DRAWINGS

FIGS. 1A and 1B show a biodisc system embodiment of the present invention, and FIG. 1B illustrates the vertical arrangement of oligonucleotide probes, not to scale, that are disposed along a spiral track on the surface of the biodisc.

FIG. 2 is a flowchart of a method for sequencing nucleic acid according to the present invention.

FIGS. 3A and 3B are logical cross-sections of a 20-base biodisc embodiment of the present invention.

FIG. 4 illustrates an apparatus suitable for implementing a method in accordance with the present invention.

FIG. 5 represents a process in making optical-disc oligomer-microarray masters, with rotating chrome blank with photoresist that is exposed with a laser beam recorder (LBR).

FIG. 6 represents a method embodiment of the present invention for making biodiscs with a SOAP-conformal image transfer method.

FIG. 7A represents a transparent, flat hollow biodisc diskette in a floppy disc type case with a slider

FIG. 7B represents a disc inside the floppy disc type case of FIG. 7A.

FIG. 7C illustrates a read head and test solution used with the apparatus of FIG. 7A.

### DETAILED DESCRIPTION

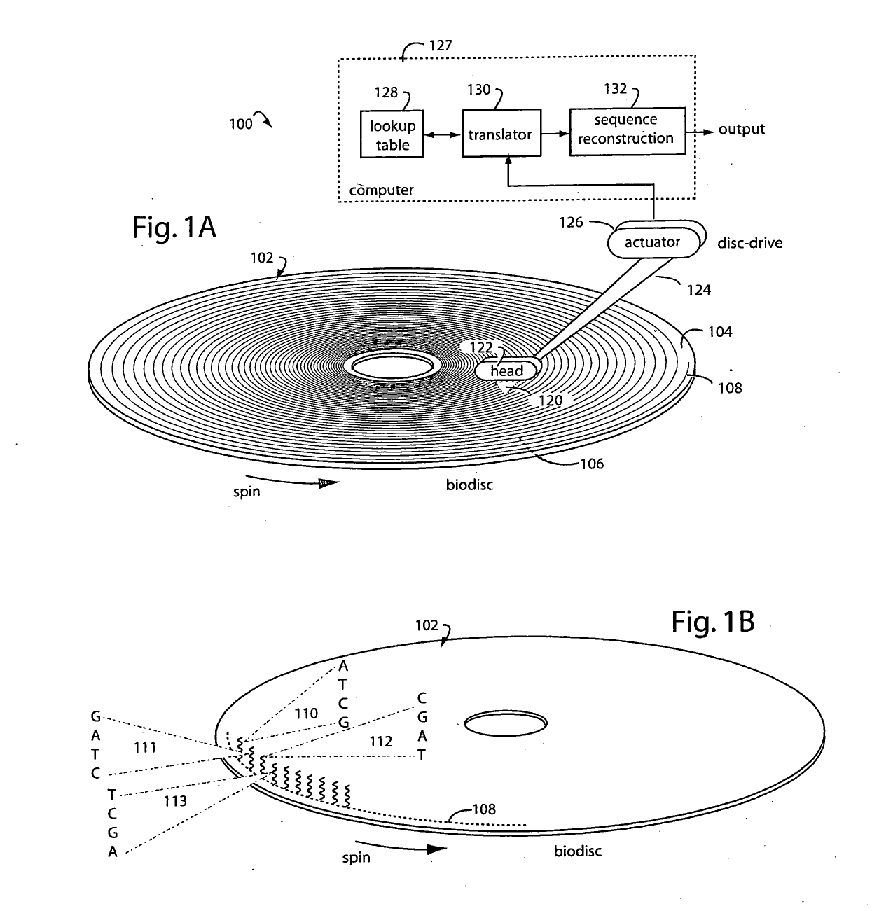

FIG. 1A illustrates a biodisc for sequencing by hybridization (SbH) in a system embodiment of the present invention, referred to herein by the reference numeral 100. The SbH system 100 includes a flat disc substrate (biodisc) 102 that resembles a compact disc (CD). Such substrate format affords many advantages in that relatively inexpensive and readily available CD mastering, duplication, and optical player technology and equipment can be used. The biodisc 102 includes opposite surfaces 104 and 106. A spiral 108 is disposed on one surface and comprises a one-dimensional array of sites with unique physical addresses. For example, the physical addresses can be referenced by the relative angle (theta) and radius (R), or by a sequential sector or block number. Embodiments of the present invention use a physical addressing scheme that is similar to those used for audio-CD and CD-ROM discs so that the corresponding disc-player technology can be adapted.

A biodisc system includes a biodisc with a variety of oligonucleotide probes with different sequences disposed on its surfaces at predetermined and addressable locations. A disc drive provides for mounting and optical scanning of the biodisc. A computer is connected to the disc drive and has a-priori information about the predetermined and addressable locations of the oligonucleotide probes disposed on the biodisc. During scanning, its software on the computer can detect any hybridization of particular ones of the oligonucleotide probes when the biodisc is mounted on the disc drive with the help of flouroluminescent tags on the samples in accordance with well known techniques and as described herein. Thus the particular genetic sequences in a sample material that were in contact with the biodisc can be identified to a researcher.

FIG. 1B illustrates four oligonucleotide probes 110-113 that are typical of those disposed along spiral 108. For sake of clarity in this example, probes are illustrated with a 4-base vertical depth (i.e., 4-mer). Probe lengths of 10-20 mer are more typical in actual practice and are suitable for use in accordance with the present invention. The illustrations of FIGS. 1A and 1B represent these probes as being corkscrew, and such physical structure is a natural occurrence in these kinds of molecules.

A synthetic-DNA probe 110 has a first physical address and comprises a base sequence, for example, of ATCG, reading from outside-to-substrate. A synthetic-DNA probe 111 has a second physical address and comprises another base sequence, for example, GATC. A synthetic-DNA probe 112 has a third address and comprises a base sequence, for example, CGAT. A synthetic-DNA probe 113 has a fourth physical address and comprises, for example, a base sequence of TCGA.

FIG. 1B merely shows one example, and practical embodiments of the present invention will have as many as a billion unique synthetic-DNA probes each with as much as a 20-base depth, e.g., giving 4 to the 20th power unique sequences(roughly 1 trillion) that can be matched.

Returning to FIG. 1A, after hybridization of a target, a light beam 120 is used by a combination light source and detector head 122 to read any light returned from hybridized nucleotide probes on the spiral 108. A motor and spindle are used to spin the biodisc 102 as in a conventional CD-type optical disc drive. An actuator 124 connects to a servo motor 126 such that the head can be positioned at any radial position relative to the biodisc 102. A computer 127 provides scanning and analysis functions. An inventory of the oligonucleotide probes is cataloged in a database according to their physical addresses on the biodisc 102. A lookup table 128 provides an index into this catalog that is used by a translator 130 to convert physical addresses on the spiral to the corresponding base-sequence in a reconstruction processor 132.

In some embodiments of the present invention, it may be advantageous to optically encode computer data that includes the information for lookup table 128 directly on the biodisc 102 using CD-ROM techniques. Such encoding can, for example, help avoid errors matching the physical synthetic-DNA addresses to the base-sequence information from the hybridization.

The DNA oligos arrays can be arranged in parallel with digitally encoded CD-tracks for servo tracking, probe markings, alignment, labeling, coding schemes, and other information. Such encodings can be used to identify the synthesized genes, the number of probes, the address location within the biodisc, part numbers, manufacturer names, etc.

In embodiments of the present invention, the overall surface area can be very large, e.g., as in a ten-inch diameter standard video-disc. Such large surface areas can be used to advantage to provide enough space for a billion or more unique synthetic-DNA probes. Fluorescent markers attached to a target sample will provide tell-tales as to how much and exactly which probes were hybridized.

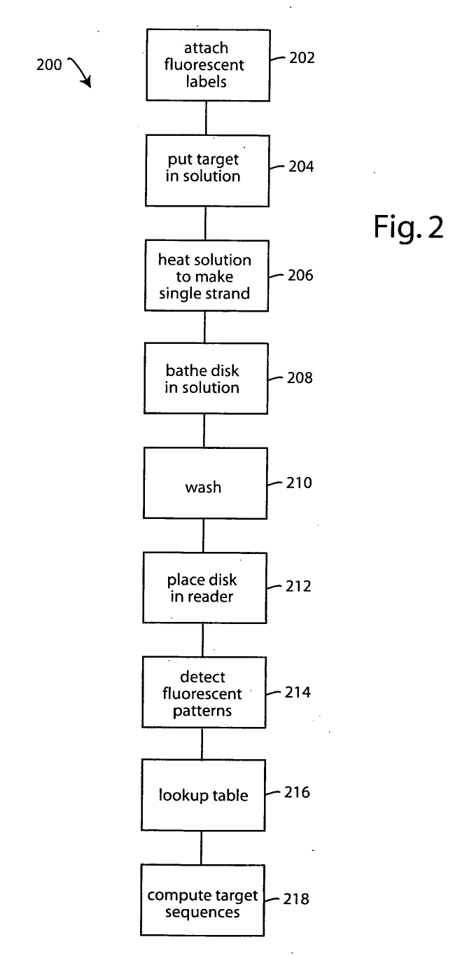

FIG. 2 represents a nucleic acid sequencing method embodiment of the present invention, and is referred to by the general reference numeral 200. In a step 202, fluorescent-labeled receptors are attached to a target sample to be analyzed. The fluorescent label can be attached covalently or by intercalation, and may comprise ethidium bromide, succinylfluoresceins, FITC, NBD, Texas Red, and tetramethylrhodamine isothiocynate. The covalent attachment can be done either chemically or enzymatically. The typical illumination source will be ultraviolet (UV), e.g., 308 nanometers (nm).Luminescence will usually be detected in the red spectrum, e.g., 630 nm. A step 204 places a target sample in solution. A step 206 heats the solution to obtain single-strand target material. A step 208 bathes a disc, such as biodisc 102 in FIGS. 1A and 1B, in the solution for hybridization. A step 210 washes away any unreacted solution.

A step 212 places the disc in a disc-player apparatus such as is shown as system 100 in FIG. 1A. A step 214 illuminates the disc in ultraviolet light and detects any red spectrum fluorescence, e.g., disc sector block-by-block until the entire longitudinal length of spiral 108 is read. A lookup table is referenced in a step 216 that translates the physical addresses that show some fluorescence into the corresponding nucleic acid sequences for the synthetic DNA-probes located at those sites. A step 218 computes the target sequences by interpolating the data from step 216. Overlaps in different sequences are used to string together sequences with bases larger in number than the disc has.

In a biodisc fabrication process, e.g., in a 20-base CD-format that is 120 mm (OD) by 35 mm (ID), as many as eight billion synthetic-DNA probes can be accommodated with spot sizes as small as 0.8 micron and both sides of the biodisc are used.

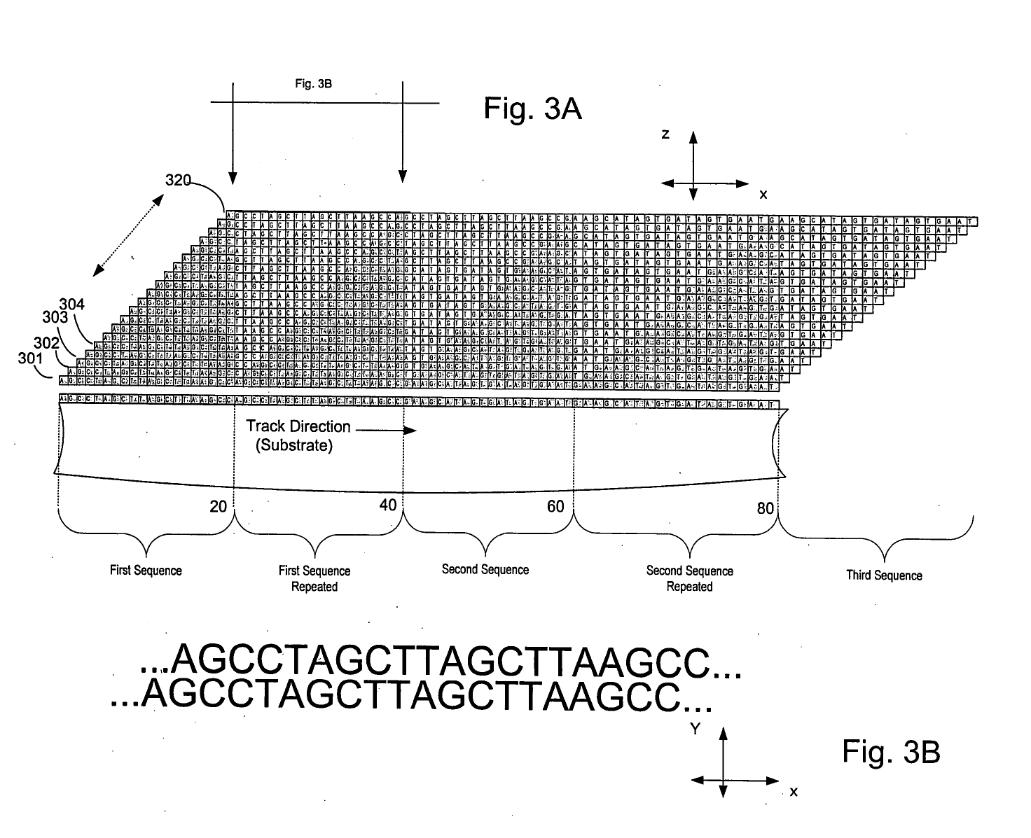

FIG. 3A is a cross-section of a 20-base biodisc embodiment of the present invention that uses only four masks in its fabrication. It will be understood that FIG. 3A represents a logical view of the biodisc, rather than a physical view. The cross section shown in FIG. 3A is in XZ plane (relative to the disc reference of FIG. 1) and the probe stack is from the substrate surface upward away from the disc, increasing in the Z direction. In this example, the X direction is the track direction.

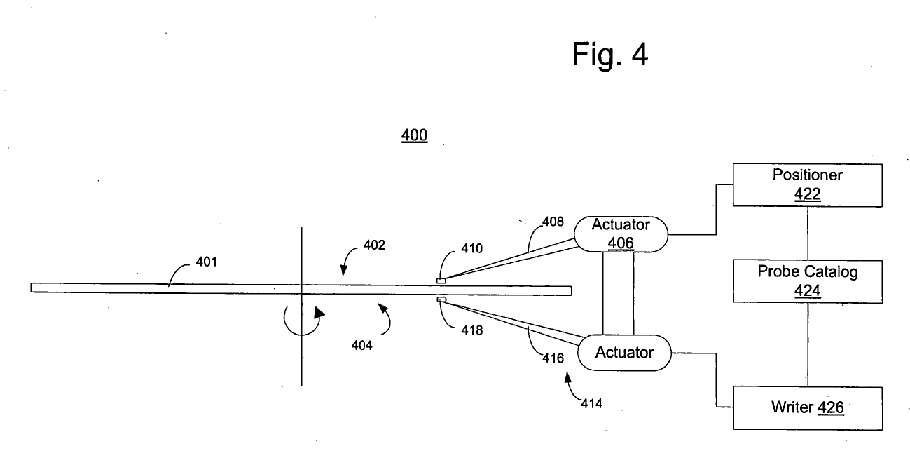

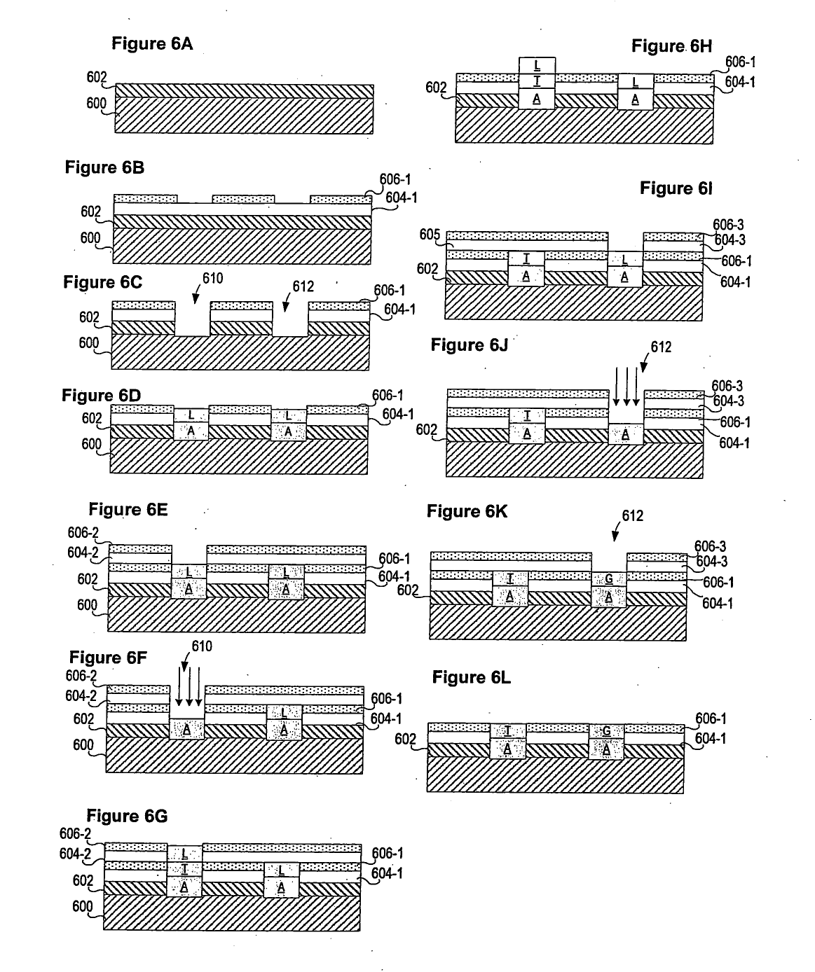

As noted above, a physical depiction of the disc is shown in FIGS. 1 and 2, where the molecules are shown sticking from the surface upward the sequence. While the addresses are shown as being adjacent in FIGS. 3A and 3B, it will be understood that test solution will reach the surface of the disc and engage each molecule at each address. The addresses are depicted in FIGS. 3A and 3B as boxes labeled according to the contents. In the boxes, all molecules are of the same sequences. However, depending on how many molecules you put in the boxes there is room for other molecules in the test solution to reach to the bottom boxes of the address sequence illustrated. It will be further understood that the logical depiction shown in FIGS. 3A and 3B may be formed by the methods discussed with respect to FIGS. 4 or 6.

In this example, two sequences are investigated. The first one is AGCCTAGCTTAGCTTMGCC and the second one is GAAGCATAGTGATAGTGAAT. It is assumed that the first and the second sequences are of some minor interest and probed twice.

Referring to FIG. 3A, a first layer 301 is deposited on the substrate of the biodisc that repeats an oligomer base pattern, e.g., “AGCCTAGCTTAGCTTAAGCC”. This pattern is deposited along the entire length of the spiral using four oligomer deposition cycles that each follow photoresist and masking, e.g., “A”, “G”, “C”, and “T”. The design of each such mask and their combination provides the information needed to preload the lookup table 128 (FIG. 1A).

Referring again to FIG. 3A, a second layer 302 repeats the pattern in the first layer 301, but shifts the oligomer base pattern “AGCCTAGCTTAGCTTAAGCC” forward by one. This overlaying and shifting forward by one is repeated for every successive layer 303 through 320. Such technique pattern shifting lowers manufacturing costs because only four masters are needed.

Master sequences are thus preferably used twenty times for 20-mer, 40 times for 40-mer, and so on. A typical synthetic-DNA probe 320 has a “AGCCTAGCTTAGCTTAAGCC” sequence. FIG. 3A shows two identical sequences. One analysis showed that total number of sequences that can be built this way is the total gross probe capacity of the biodisc divided by 20. Only one probe sequence is completely functional with the desired sequence, while the remaining nineteen are only partially hybridizable. As a result, the variation of both hybridization and hence the fluorescence intensity resembles a saw-tooth intensity distribution graph (illustrated by the shading of FIG. 3A, with a leading edge up to address twenty and drop off gradually at the twenty first address, then a climb back up to a second peak at forty, at which point, hybridization is complete and stable again.

In this manner, millions of probes can be accommodated on a biodisc by using only four masks repeated twenty times. The total number of available sequences is the total number of bits (pits) divided by 20.

Each layer in a biodisc can be produced with four exposure and depositions cycles, e.g., one for each of A, G, T, and C. For example, at an arbitrary starting address of “1”, a first layer has a “AGCCTAGCTTAGACTT” pattern deposited. Table I summarizes the four mask patterns that are needed to do this.

TABLE I

| layer-1 | A | G | C | C | T | A | G | C | T | T | A | G | A | C | T | T |
|---|---|---|---|---|---|---|---|---|---|---|---|---|---|---|---|---|
| Mask-A | x | | | | | x | | | | | x | | x | | | |
| Mask-G | | x | | | | | x | | | | | x | | | | |
| Mask-C | | | x | x | | | | X | | | | | | x | | |
| Mask-T | | | | | x | | | | x | x | | | | | x | x |

Table II shows the pattern for layer-2 relative to address “1”. This pattern is easily implemented by moving the substrate one position to the left. Compare Tables I and II.

TABLE II

| layer-2 | T | A | G | C | C | T | A | G | C | T | T | A | G | A | C | T | T |
|---|---|---|---|---|---|---|---|---|---|---|---|---|---|---|---|---|---|
| Mask-A | | x | | | | | x | | | | | x | | x | | | |
| Mask-G | | | x | | | | | x | | | | | x | | | | |
| Mask-C | | | | x | x | | | | x | | | | | | x | | |
| Mask-T | x | | | | | x | | | | x | x | | | | | x | x |

Table III shows the pattern for layer-3 relative to address “1”. This pattern is also easily implemented by advancing the substrate (disc) one more step to the left. Compare Tables II and III.

TABLE III

| layer-3 | T | T | A | G | C | C | T | A | G | C | T | T | A | G | A | C | T | T |
|---|---|---|---|---|---|---|---|---|---|---|---|---|---|---|---|---|---|---|
| Mask-A | | | x | | | | | x | | | | | x | | x | | | |
| Mask-G | | | | x | | | | | X | | | | | x | | | | |
| Mask-C | | | | | x | x | | | | x | | | | | | x | | |
| Mask-T | x | x | | | | | x | | | | x | x | | | | | x | x |

In summary, four-mask patterning and shifting method embodiments of the present invention greatly reduce the number of total masks required in biodisc manufacturing. A standard CD has a recordable area of around one hundred square centimeters. If four square microns are needed per address, two billion such addresses will fit on a standard CD sized substrate. With twenty addresses per usable probe, one hundred million usable probes can be realized with only four stampers or masks, e.g., one for each base.

The feasibility of such a technique is attributable to the use of spiral CD format and would not be suitable for the Genechip-type application as such applications are based of on a square two-dimensional format. In theory, some errors are accepted in order to minimize mask costs. On the other hand, it is valuable as a low cost substitute when gene analyses become widely used, not as a specialty but as a routine procedure at the consumer level.

Biodisc embodiments of the present invention can use conventional constant angular velocity (CAV) or constant linear velocity(CLV) recording formats, as well as concentric and spiral track arrangements. It is apparent from all previous discussion that the preferred embodiment of the current invention would be CLV with constant address sizes and spiral tracks. This is considered as a natural coordinate for DNA that is basically a long linear biopolymer. As matter of fact, one spiral track can be a complete human genome, from the head to the tail in one single spiral track. The current invention would allow the complete analysis of genome in one single disc, made with only 4 masks.

It is interesting to note that whereas the original hybridization data would be zero intensity for negative and saw tooth shape for positive, the original data can be integrated or differentiated by clever electronic system design. After such data handling, it is obvious that integration leads to zero for negative and step function for positive. The differentiation is actually more interesting from the fact that the leading edge is of constant slope and the falling edge is a delta function. Such a signal would improve the detection of the precise pixel where sequence is changed from one to the other. It is therefore very useful to examine not only the original signal but the derivative signal as well.

FIG. 4 represents an in-situ probe synthesizing system 400. A highly repeatable addressing of the probes on a biodisc being fabricated is needed, so the in-situ probe synthesizing system 400 includes a fiducial disc 401 with an address index spiral 402. These parallel an in-situ biodisc 403 that has a oligonucleotide probe spiral 404 that is to be synthesized. Both tracks are shown here to be on different side of the same disc but they can also be interlaced on same side. An important factor here is the use of dual pickup head and dual tracks, one for alignment and registration and the other for synthesis of oligos.

An actuator 406 moves a servo tracking arm 408 and read head 410. A light beam 412 is used to track the address index spiral 402. A follower 414 exactly tracks actuator 406 with a tracking arm 416 and write head 418. A laser 420 writes patterns on biodisc 403 for later deposition of the four base materials, ATCG. A positioner 422 converts the electronic commands from a probe catalog 424 into disc addresses. The probe catalog 424 maps the inventory of oligonucleotide probes needed to be written onto biodisc 403 by their assigned physical addresses. Such database of catalog information is later used by scanners to interpret the results of hybridization. A writer 426 electronically drives write head 418 according to which base material is needed at any one spot, probe by probe.

The system of FIG. 4 allows an improvement in the alignment and registration during the photo lithographic manufacturing of a biodisc. Here fiducial marks used for alignment can be read by a first pick-up head (410) and the second pick-up (418) is used to expose (generate) patterns along the track. Notice that multiple exposures are required and therefore, each exposure (or depositing layers) must be properly aligned with the previous exposure (or last depositing layers). The fiducials serve as the constant template to which the disc is aligned either locally or globally for both overlay and registration. It should also be noted that the twp tracks and two pick-up heads are shown here to be on each side of the disc. In reality they can be placed on same side of the substrate. In this lafte embodiment, the disk could contain one reference track and one manufacturing track, or one regular track and one bio-track, with the regular track for tracking and biotrack for actual hybridization. Such a dual track design improves the accuracy of placement, and hence, the quality of the biodisc.

It is also possible to etch deep into the underlying glass substrates and make the necessary tracks and channels. Such tracks can be etched deep enough to accommodate all 20-mer of a probe inside each pit along the track channel.

System 400 is used for in-situ synthesis of oligomers on a biodisc. The servo master track 401 provides for highly repeatable addressing of new biotrack 403 on which the in-situ oligomers being synthesized. Such arrangement allows the recording head to revisit the same locations on the new bio track with very high accuracy and repeatability. This is necessary to be able to correctly build up the nucleotide probe sequences that need separate recording exposure passes for each A, T, C, and G component. Alternatively, the disc can be a two-sided configuration with servo/alignment tracks on one side for the read/tracking head and the synthesized oligomers on the other side accessible to the recording head. And, in another alternative embodiment of the invention, the information stored on disks 401 and 403 can be one combined into a single disc. The result is interlaced tracks and a simpler rendition with dual pick-up heads, one for tracking and one for recording. In one embodiment. This embodiment would yield a production uniformity during manufacturing. It will be understood that certain modifications would be needed to guarantee both registration, alignment and final overall overlay accuracies.

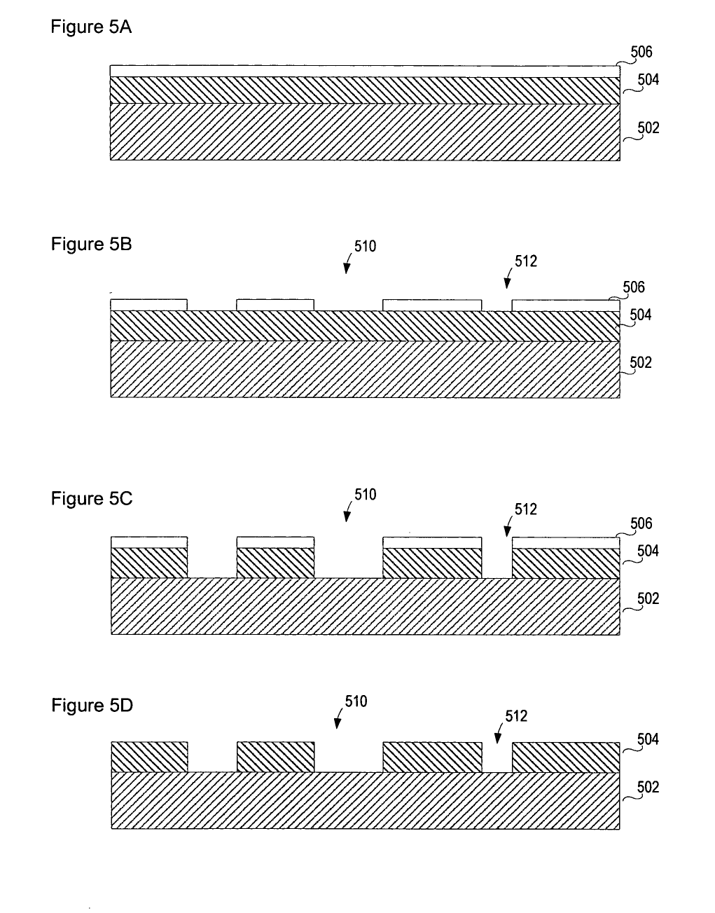

FIGS. 5a-5d represent a process for making optical-disc oligomer-microarray master discs using a rotating chrome blank with a photoresist that is exposed with a laser beam recorder (LBR). This process is similar to the standard stamper used in compact disc manufacturing. However, instead of the resist coated glass, the master is a circular chrome blank with resist, commonly used in the industry.

FIG. 5a shows a first step for depositing a photoresist 506 on a circular glass disc 502 that has been deposited with chromium film (504). 502 is placed in a laser beam recorder with a rotating spindle chuck. Techniques for depositing a photoresist and chrome layers are well known in the art. Next, FIG. 5B, laser beams modulated by piezo-electric or acoustic methods expose regions of the photoresist. Photoresist images are developed afterwards to expose underlying regions of the chromium layer 504 in regions 510, 512. In FIG. 5C, the chromium layer is etched using the remaining photoresist as a mask. Finally, as shown in FIG. 5D, resist 506 is stripped, and the remaining patterned chrome becomes a stamper that matches the original image written by the LBR. Duplicates of each master disk can then be mass produced using these chrome masters, e.g., as in conventional semiconductor manufacturing processes. These master discs can be used to duplicate biodiscs using conventional projection printing with no reduction. This is a 1:1 process. Duplicates can also be made using a step-and-repeat process, e.g., 5× or 10×.

Alternatively, in compact disc manufacturing, traditional circular masters are commonly known as stampers. They are preferably made using a laser beam recorder exposure system. The desired pattern is recorded by modulating the laser beam. The laser exposes patterns on the photoresist-coated glass substrate (with no chrome layer). Subsequent development of the latent image results in resist images. The resist layer is coated with thin silver layer, placed in a nickel tank and a nickel thin layer is grown over the resist image. The nickel stamper is removed and the resist is stripped. The stamper can be used thousands, and even millions times to produce duplicates that resemble regular compact discs.

Duplication in compact disc manufacturing is somewhat different from semiconductor manufacturing. Instead of photolithography as the prevailing technology in chip making, injection molding with stamper has been the main process. Only recently, other process such as SOAP is proposed that avoids the traditional method of injection molding with high pressure and high temperature. The SOAP CIT process will be discussed further later in FIG. 6.

A method embodiment of the present invention can be used to make duplicates with a combination of the SOAP and conformal image transfer processes. Such is similar to 1X contact process used in making sub-masters from masters. No reduction is possible, but small features can be successfully replicated. The SOAP CIT process utilizes spin coating technology for duplication via the following steps: (1) the masters are coated with a polymeric solution (2) dry or cure the solution to form a film (3) the film is peeled off, sometime via a glue to a substrate (4) features in the master is now duplicated on the SOAP film attached to the substrates. Such an image transfer is conformal so it is referred to as SOAP CIT (conformal image transfer). Prior art using SOAP in the making of DVD disc can be found in patents issued to the present inventor, including U.S. Pat. Nos. 5,468,324, 5,635,114, 5,663,016, 5,688,447, 5,700,539 and 5,846,627.

In another embodiment of the invention, a release layer is deposited in between the glue layer and the master. In which case, the release material will be transferred to the substrate. It is also possible to have a material that will stay with the master and not be transferred and is preferable because the release layer can be reused over and over again. For example, thin sputtered gold layer is inert and non-sticking.

An alternative method embodiment of the present invention uses SOAP-conformal image transfer in making biodiscs. The image from master is replicated and used as conformal flood exposure masks. The protected area will not be subject to chemical addition but the unprotected area will become active for another addition. Alternatively, the image can be used as a stencil for adding another base. The chemical addition is done at uncovered area. After stripping the first SOAP-conformal image transfer layer, the process is repeated and chemical addition at the unprotected area can be done. The SOAP layer is a chemical mask, instead of a photo mask. Results are similar, the differentiation is based on optical and chemical and photochemical properties of the SOAP-conformal image transfer layers.

The circular-format biodisc duplicates can be manufactured using conventional semiconductor and compact disc techniques. Once a master is in-hand, conventional photolithographic equipment can be used to make duplicates. Full size 1:1 projection and step-and-repeat reduction methods can be used for biodisc embodiments of the present invention. How to use photolithographic and light directed probe synthesis is straightforward. These are non-contact methods of making biodiscs.

The word “contact” only refers to the close proximity between the master and its duplicate. In the usual sense, contact printing means the master and duplicate are in actual contact and a vacuum is applied to remove any gap between. But the uneven surfaces cause the contact to be less than perfect, so errors result. Method embodiments of the present invention are different in this respect, they use a near perfect contact in which gaps between the master and its duplicate do not exist.

FIGS. 6a-6l represent a method in accordance with the present invention for making biodiscs with a SOAP-conformal image transfer method. As shown at FIG. 6a, a substrate 600 has a reflective chromium layer 602 deposited thereon. In one embodiment, reflective chrome layer 602 is deposited on a glass substrate 600. In alternative embodiments, a light-absorbing layer is used to replace the chrome so that a dark background will be provided to help contrast fluorescent-dye marked hybridization sites of the oligonucleotide probes being formed.

Next, as shown in FIG. 6B, a spin-on-and-peel conformal image transfer (SOAP-CIT) layer 606-1 is placed on the chromium layer 602 and will be used to define lands and pits In FIG. 6B, the glue layer 604-1 is also shown. Layer 604-1 was incorporated into the peel off process to transfer 606-1 from the master to the chrome and glass substrate (600 and 602). Next, using layer 606-1 as a masking layer, an etch process is used to etch away portions of the glue layer 604-1 and reflector 602 to open pits 610, 612. As shown at FIG. 6d, a base, e.g., “A”, is deposited in the pits and capped with a protective layer “L”. Similar processes lead to the structure shown at FIG. 6E, as second conformal image transfer layer 606-2 and glue layer 604-2 are deposited, and flood exposure is used to remove the protective layer “L”, at a first address only, e.g. pit 610, but not at pit 612. As shown in FIG. 6f, another base, “T”, is deposited on the first address with chemistry. At FIG. 6g, base “T” is covered with a protective layer “L”. Next, as shown in FIG. 6h, the layer 606-2 and layer 604-2 are stripped. Next, as shown in FIG. 6l, another CIT layer 606-3 and 604-3 is deposited and used to expose the second address (pit 612) and simultaneously protect the first address (pit 610). As shown in FIG. 6j, the protective layer in pit 612 is removed at flood exposure and, as shown in FIG. 6k, a base, “G”, is added to the second address. Next, as shown in FIG. 6l layers 606-3 604-3 are stripped. The process is used repeatedly to construct the desired oligo sequences.

At this point, the pits are aligned along a track and inter-digited with groove channels in a traditional compact disc format. A land area straddles both sides of the along-track string of grooves and pits, and runs parallel in between pits. Such land areas separate adjacent tracks from one another. The oligonucleotide probes are thus being constructed in the pits and are separated from each other along-track by the grooveland areas. It should be recognized that repetition of the previous steps occurs in building additional layers, G at the first and A at the second address until all layers of the probe are constructed.

The image from a master is replicated and used as a flood exposure mask as discussed with respect to FIG. 6e and 6i above. The protected area will not be subject to chemical addition but the unprotected area will become active for another addition. In another variation of the invention, the image can be used as a stencil for adding another base. The chemical addition is done at uncovered area. After stripping the first SOAP-conformal image transfer layer, the process is repeated and chemical addition at the unprotected area can be done. In this usage, the SOAP layer is a chemical mask, instead of a photo mask. Results are similar, the differentiation is based on optical and chemical and photochemical properties of the SOAP-conformal image transfer layers. Certain material will be used as a chemical mask whereas others might be used as a photo mask. Alternatively, the image from a master can be replicated through the traditional photo lithographic process in the well-known art of chip processing, independent of the SOAP-CIT process proposed here. Compared to conventional photolithographic method, the current method does not require the use of photo-sensitive material and also eliminating the conventional developing step. More flexibility and wider choice of material and process is a distinct advantage of the present invention.

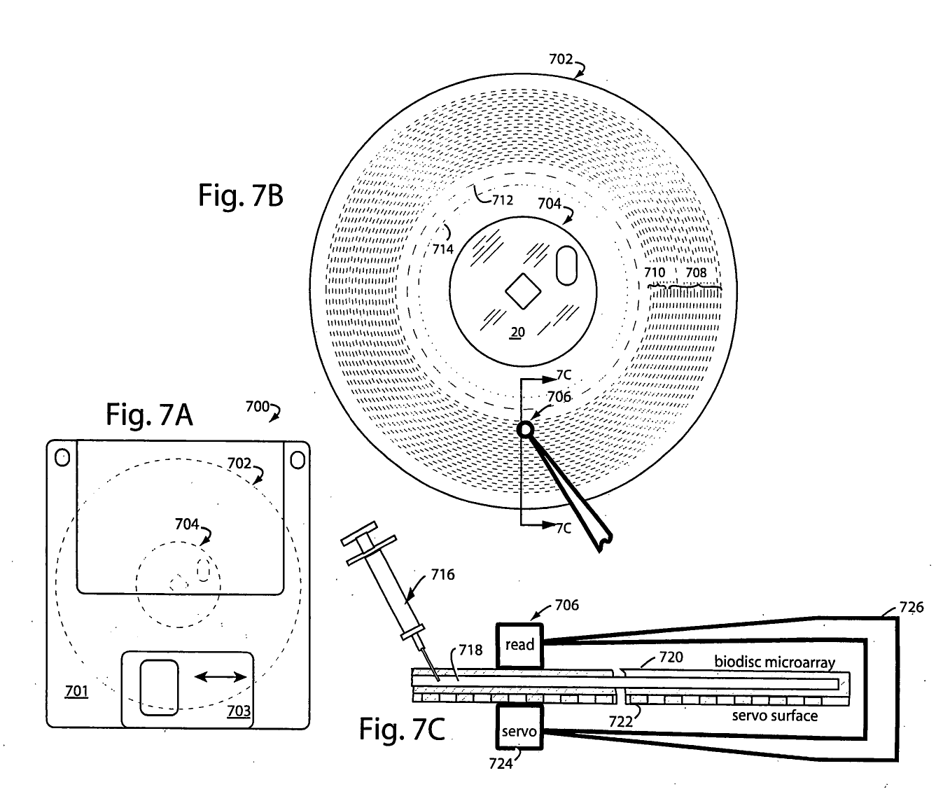

FIG. 7A represents a transparent, flat hollow diskette in a floppy disc type case with a slider door, and in which microarrays of oligonucleotide probes are disposed on the inside surfaces of the hollow, and so that samples can wash by, hybridize and their attachments be optically viewable from outside through the slider door. Notice that the possibility of spinning the disc not only makes it easier to read and write but also might help in hybridization. The chemical reaction can be accelerated with spinning also. A sample is placed inside the cavity and is in contact with the micro-array for hybridization. The reading can be done with the pickup up head, through the glass disc or other transparent protective plastics, much the same way the ordinary compact disc is read.

The complete assembly (700) resembles a typical floppy disc as shown in FIG. 7A. It is use here as a convenient illustration. Similar mechanical arrangement is used for compact discs also. The general designing principle involved is quite similar. A biodisc (702) is placed within a protective casing (701) for cleanliness with a sliding door (703) and a hub (704) that will be engaged with a rotating motor. The detailed way of engaging the disc is immaterial, for example, pneumatic, magnetic, etc. can all be employed. An optical pickup head (706) is shown in FIG. 7B and detailed arrangement shown in FIG. 7C. A biodisc (702) with probes (708,710) are deposited along the tracks. Additional labeling or cataloging marks, instructions, serial numbers, etc. can be included for identification (712). Alignment and/or registration marks for tracking can also be placed on the same disc (714). A cavity is built-in for injection of samples with a syringe (716) and can be sealed off or reopen for washing. To reduce the bubbles, multiple inlets or outlets may be needed. Microarrays (720) are deposited in the insides of the hollow. It can be one-sided or double sided (both upper side and lower side. The sample (718) within the cell will come in close contact with the microarray for hybridization. A simple pickup head with servo (724) for centering is shown here. The servo read information at the bottom (722) as shown in FIG. 7C. More elaborated design with multiple read heads (for example, one pick up head for upper microarray and the other pickup head for lower, etc. ) can be used. The glass or plastic substrates supporting the micro array is not shown here. It is obvious that micro array must be manufacture with processes, including but not limited to, the SOAP process (FIG. 6 ). The cells are, then, assembled. The completed cell might have to be designed to tolerate certain thermal stress that are required for hybridization proposes. These are additional design constraints that can be accommodated by proper use of material with suitable chemical, mechanical, thermal and optical stabilities.

Although particular embodiments of the present invention have been described and illustrated, such is not intended to limit the invention. Modifications and changes will no doubt become apparent to those skilled in the art, and it is intended that the invention only be limited by the scope of the appended claims.

The foregoing detailed description of the invention has been presented for purposes of illustration and description. It is not intended to be exhaustive or to limit the invention to the precise form disclosed. Many modifications and variations are possible in light of the above teaching. The described embodiments were chosen in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

I claim:

1. A biodisc, comprising:
a circular disc having a concentric middle blank area for a spindle and opposing planar sides; and
a number of oligonucleotide probes with different sequences disposed along tracks at addressable locations on at least one of said opposite opposing flat planar sides.

2. The biodisc of claim 1, further comprising:
a number of servo tracks on one or the other said opposite flat planar sides and providing for repeatability in addressing of individual ones of the number of oligonucleotide probes disposed on a one or the other of said opposite flat planar sides.

3. A biodisc system, comprising:
a biodisc with a variety of oligonucleotide probes with different sequences disposed on its surfaces at predetermined and addressable locations;
a disc drive for mounting and optical scanning of the biodisc; and
a computer connected to the disc drive and having a-priori information about said predetermined and addressable locations of said oligonucleotide probes disposed on the biodisc, and able to detect any hybridization of particular ones of said oligonucleotide probes when the biodisc is mounted on the disc drive, and further able to thereby identify particular genetic sequences in a sample material that was in contact with the biodisc.

4. A method for manufacturing a in-situ synthesized biodisc, comprising:
coupling together a servo master disc and an in-situ biodisc together on a common spindle;
paralleling a read head for tracking position information on said servo master disc, and a writing head for recording oligonucleotide information on said in-situ biodisc together on a common actuator;
determining a particular oligonucleotide for synthesis at a location on the in-situ biodisc;
addressing each location on said in-situ biodisc indirectly with said read head for tracking position information on said servo master disc;
addressing said locations on said in-situ biodisc as said particular oligonucleotide is in the process of being synthesized.

5. An method for manufacturing a in-situ synthesized biodisc, as describe in claim 4 whereby the master and the and the replica is the same disc and reading tracks and writing tracks are interlaced on one and the same side of the said flat planar sides.

6. An method for manufacturing a in-situ synthesized biodisc, as describe in claim 4 whereby the master and the and the replica is the same disc and reading tracks and writing tracks are located on opposite sides of the said flat planar sides.

7. A mass-production method for manufacturing a biodisc, comprising:
using only four different masks in shifted angular positions during each of a plurality of masking operations to synthesize oligonucleotide sequences on a biodisc that exceed four-mer.

8. An biodisc, comprising:
a flat planar disc substrate;
a spiral disposed on at least one surface of the disc substrate;
a first oligomer deposited at a first physical address in the spiral and having an affinity during hybridization for a first DNA fragment; and
a second oligomer deposited at a second physical address in the spiral and having an affinity during hybridization for a second DNA fragment.

9. The biodisc of claim 8, wherein:
the first and second oligomers and the first and second physical addresses occur in a particular sequence.

10. The biodisc of claim 8, further comprising:
a first DNA fragment hybridized to the first oligomer. The DNA fragment has been dyed with a fluorescent; and
a second DNA fragment hybridized to the second oligomer. The second fragment has been dyed with a fluorescent.

11. The biodisc of claim 10, wherein:
the first and second physical addresses occur in a particular sequence and a fluorescent glow from these addresses is evidence of a hybridization of a DNA fragment with both oligomer probes. The double positive is an evidence of a particular DNA sequence.

12. A nucleic acid sequencing method, comprising the steps of:
attaching fluorescent-labeled receptors to a target sample to be analyzed;
placing said target sample in a solution;
heating said solution to obtain a single-strand target material;
hybridizing a biodisc in said solution;
washing away any unreacted solution;
reading patterns of hybridization as indicated by said fluorescent-labeled receptors present at particular physical addresses while rotating said biodisc, and scanning radially across with a detector head until an entire longitudinal length of a synthetic-DNA spiral on a surface of said biodisc is read;
using a lookup table to translates a detected physical address into a complementary nucleic acid sequence for a corresponding synthetic DNA-probe; and
computing a target sequence by interpolating data obtained by from said lookup table.

13. A method for fabricating a biodisc that has a spiral line of synthetic-DNA probes, the method comprising the steps of:
depositing a first layer on a biodisc substrate that repeats a oligomer base pattern along an entire length of the spiral line using four oligomer deposition cycles “A”, “G”, “C”, and “T”;
depositing a second layer that repeats said oligomer base pattern on top of said spiral line and said first layer, but shifts the oligomer base pattern forward by one probe; and
depositing n-number of layers that repeat said oligomer base pattern on top of said spiral line and a lower layer, but shifts the oligomer base pattern forward by one;
wherein, at least n uniquely sequenced synthetic-DNA probes each with n-base sequences are fabricated on said substrate for hybridization of nucleic acid target samples.

14. A biodisc for hybridization of nucleic acid target samples, comprising:
a flat, round disc substrate for optical scanning in a CD-type disc drive;
a plurality of independent and individually unique synthetic-DNA probes deposited on the disc substrate in a single layer comprising A, T, C, and G affinity oligomers;

15. The biodisc of claim 14, wherein:
plurality of independent and unique synthetic-DNA probes have at least two subclasses of with different synthetic-DNA probes numbered base sequences.

16. The biodisc of claim 14, wherein:
the flat disc substrate has an outside diameter of at least ten inches; and
the plurality of independent and unique synthetic-DNA probes are deposited in four different masking cycles, one for each of A, T, C, and G.

17. A biodisc assembly, comprising:
a circular substrate having a concentric middle blank area for a spindle and opposing planar sides;
a number of oligonucleotide probes with different sequences disposed along tracks at addressable locations on at least one of said opposite opposing flat planar side; and
an enclosure having samples to be analyzed by hybridization

18. The biodisc of claim 17 wherein the assembly is scannable from outside through transparent substrates.

19. A system, comprising:
a biodisc assembly, including a circular substrate having a concentric middle blank area for a spindle and opposing planar sides and a number of oligonucleotide probes with different sequences disposed along tracks at addressable locations on at least one of said opposite opposing flat planar side; and
an enclosure having samples to be analyzed by hybridization;
a read head and detection electronics for detecting hybridization and analysis.
